(19) United States
(12) Patent Application Publication
(10) Pub. No.: US 2025/0032021 A1
(43) Pub. Date: Jan. 30, 2025

(54) STRESS DETECTION APPARATUS, STRESS DETECTION METHOD, AND STORAGE MEDIUM STORING STRESS DETECTION PROGRAM

(71) Applicant: KABUSHIKI KAISHA TOSHIBA, Tokyo (JP)

(72) Inventors: Takashi SASAKI, Yokohama Kanagawa (JP); Takashi SUDO, Fuchu Tokyo (JP); Mami SAITO, Fujisawa Kanagawa (JP); Tsukasa IKE, Tokyo (JP); Izumi FUKUNAGA, Tokyo (JP); Tomoya TSURUYAMA, Kawasaki Kanagawa (JP)

(73) Assignee: KABUSHIKI KAISHA TOSHIBA, Tokyo (JP)

(21) Appl. No.: 18/591,420

(22) Filed: Feb. 29, 2024

(30) Foreign Application Priority Data
Jul. 25, 2023 (JP) 2023-120861

(51) Int. Cl. A61B 5/16; A61B 5/00; A61B 5/0205; A61B 5/024; A61B 5/352

(52) U.S. Cl. CPC A61B 5/165; A61B 5/0205; A61B 5/352; A61B 5/4035; A61B 5/02405

(57) ABSTRACT

According to one embodiment, a stress detection apparatus includes a processor. The processor acquires heartbeat information. The processor calculates a heart rate variability factor from the heartbeat information. The processor detects change of an activity of sympathetic nervous system, based on the heart rate variability factor. The processor detects change of an activity of parasympathetic nervous system, based on the heart rate variability factor. The processor detects presence/absence of a stress state, based on time from detection of the change of the sympathetic nervous system to detection of the change of the activity of the parasympathetic nervous system. The processor acquires outside stimuli. The processor identifies a stressor that has provided the stress state from the acquired outside stimuli, based on timing at which the change of the sympathetic nervous system has been detected if the stress state is detected.

### CROSS-REFERENCE TO RELATED APPLICATION

This application is based upon and claims the benefit of priority from Japanese Patent Application No. 2023-120861, filed Jul. 25, 2023, the entire contents of which are incorporated herein by reference.

### FIELD

Embodiments described herein relate generally to a stress detection apparatus, a stress detection method, and a storage medium.

### BACKGROUND

Generally, mental health disorders, such as depression, are caused by long-term exposure to strong stress. For this reason, if the stress state and the cause of the stress are grasped by continuous observation of the mental state, there is high possibility that measures can be taken in advance against mental health disorders.

As a method for grasping the stress state of human, a known method is a method of detecting a stress state using LF and HF serving as heart rate variability (HRV) factors that calculated from heartbeat of the subject.

Conventional stress detection methods are capable of evaluating presence/absence of stress in measurement and the degree of the stress. However, conventional stress detection methods have difficulty in evaluation of elements relating to protection of mental health, such as recognition of stressors and evaluation of influences of the stressors.

### BRIEF DESCRIPTION OF THE DRAWINGS

FIG. 1 is a diagram illustrating a configuration of an example of a stress detection apparatus according to an embodiment.

FIG. 2 is a diagram illustrating a general electrocardiographic waveform.

FIG. 3A is a diagram illustrating RRI data serving as heartbeat interval data.

FIG. 3B is a diagram illustrating interpolated RRI data.

FIG. 3C is a diagram illustrating LF, RF, and LF/HF after heart rate variability factors are calculated.

FIG. 4 is a diagram illustrating autonomous nervous system activities occurring by stimuli from an outside environment, based on EGMsasa.

FIG. 5 is a diagram illustrating a sympathetic nervous system activity power SN, a parasympathetic nervous system activity power PSN1, and a parasympathetic nervous system activity power PSN2 painted in different hatching.

FIG. 6 is a diagram illustrating an example of a hardware configuration of the stress detection apparatus.

FIG. 7 is a flowchart illustrating operations of the stress detection apparatus.

FIG. 8 is a diagram illustrating chronological changes of RRI, HF, LF, and LF/HF as experimental results.

### DETAILED DESCRIPTION

In general, according to one embodiment, a stress detection apparatus includes a processor including hardware. The processor acquires heartbeat information of a subject. The processor calculates HRV factors from the heartbeat information. The processor detects change of an activity of sympathetic nervous system of the subject, based on the HRV factors. The processor detects change of an activity of parasympathetic nervous system of the subject, based on the HRV factors. The processor detects presence/absence of a stress state of the subject, based on time from detection of the change of the sympathetic nervous system to detection of the change of the activity of the parasympathetic nervous system. The processor acquires outside stimuli to the subject. The processor identifies a stressor that has provided the stress state to the subject from the acquired outside stimuli, based on timing at which the change of the activity of sympathetic nervous system has been detected if the stress state is detected.

Humans are exposed to stressors. Also, the degree of stress felt varies from person to person. The influence of the stressor on the human also differs from person to person. For such an object having no generality, to grasp the stressor felt by individuals and the degree of influence thereof is equal to provide a general solution that what stress is.

The technique explained in an embodiment is a solution derived from a discovery of a mechanism that human emotions are caused by autonomous nervous system activities and a conclusion, which is derived therefrom, that emotions are mental expression types occurring in sympathetic nervous system activity. In this manner, a stress detection apparatus according to an embodiment is capable of identifying a stressor when it detects a state in which the subject is under stress, and calculating the degree of influence provided by the stressor on the individual. By grasping the stressor and the degree of influence thereof, the stress detection apparatus according to the embodiment can contribute to protection for humans against stress disorder.

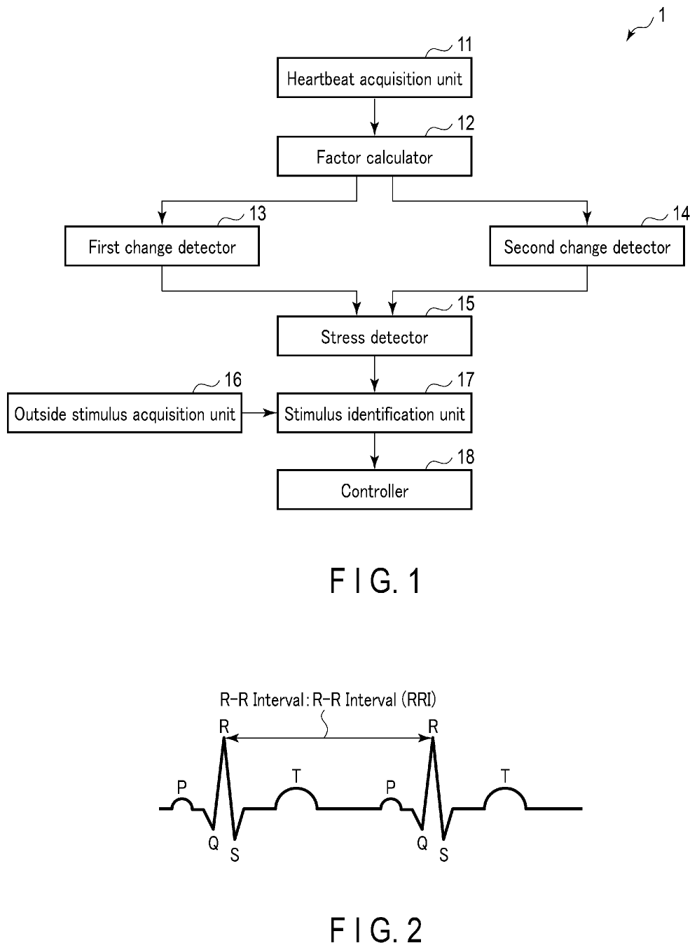

An embodiment will be explained hereinafter with reference to drawings. FIG. 1 is a diagram illustrating a configuration of an example of a stress detection apparatus according to the embodiment. A stress detection apparatus 1 is a computer including a heartbeat acquisition unit 11, a factor calculator 12, a first change detector 13, a second change detector 14, a stress detector 15, an outside stimulus acquisition unit 16, a stimulus identification unit 17, and a controller 18.

The heartbeat acquisition unit 11 acquires heartbeat information of the subject. The heartbeat information can be heartbeat interval data. FIG. 2 illustrates a general electrocardiographic waveform. Generally, A P wave occurs by ignition of a sinus node serving as a time keeper for heartbeat, and thereafter Q, R, and S waves indicating the process of excitation from the cardiac atrium to the entire cardiac ventricle occur as a complex wave. The heartbeat interval data is, for example, time intervals (R-R intervals: RRI) between R waves and subsequent R waves. The heartbeat acquisition unit 11 acquires, for example, data of intervals between R waves and R waves serving as heartbeat interval data from electrocardiographic data acquired from an electrocardiograph. Cardiac beat of the heart continuously fluctuates. Fluctuations of the cardiac beat of the heart reflects activities of autonomous nervous system. Activities of the autonomous nervous system are influenced by not only stimuli from the outside but also various mental factors, such as internal factors.

The factor calculator 12 calculates HRV factors from the heartbeat interval data. In the embodiment, the factor calculator 12 calculates, as HRV factors, LF (power of low frequency) serving as a low frequency domain component of a frequency domain of the HRV data, HF (power of high frequency) serving as a high frequency domain component thereof, and LF/HF serving as a quotient of LF and HF. LF is supposed to indicate the total activity of the sympathetic nervous system activity and the parasympathetic nervous system activity. HF is supposed to indicate the parasympathetic nervous system activity. LF/HF is supposed to indicate the activity of only the sympathetic nervous system.

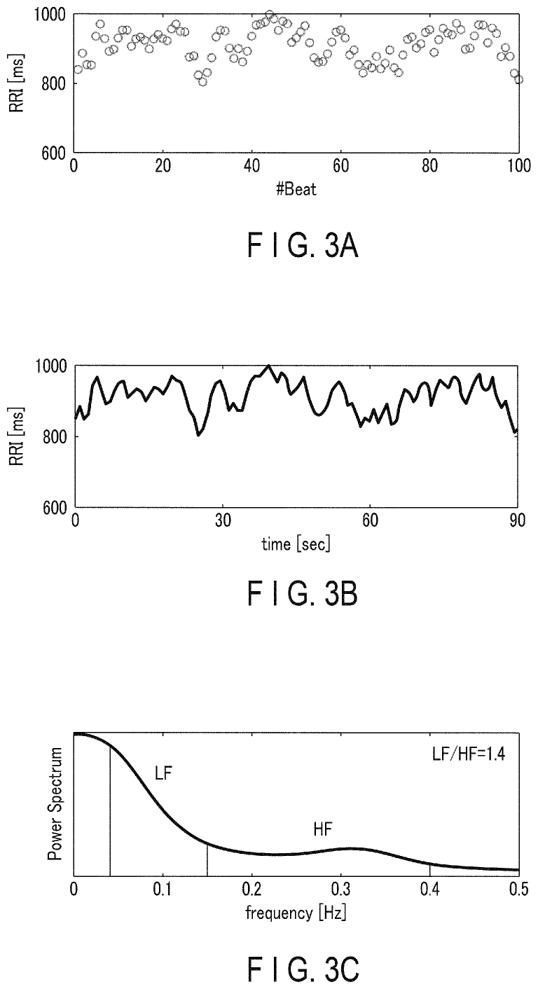

The following is an explanation of an example of calculation of the HRV factors by the factor calculator 12. For example, suppose that the heartbeat acquisition unit 11 has acquired RRI data serving as the heartbeat interval data as illustrated in FIG. 3A. As described above, because the heartbeat continuously fluctuates, the value of RRI does not have a fixed value. RRI data is converted into a frequency spectrum by Fourier transform (Fast Fourier Transform), but generally the number of measurement points of RRI data often fails to supply a sufficient number of samples to execute FFT with high accuracy. For this reason, the factor calculator 12 increases the number of apparent points of data by executing linear interpolation for the RRI data, as illustrated in FIG. 3B. Thereafter, the factor calculator 12 executes Fourier transform for the linear-interpolated RRI data to calculate power spectral density of the RRI data in the frequency space. FIG. 3C illustrates the power spectral density of the RRI data. LF is the sum total of the power in the low frequency region (0.04 Hz to 0.15 Hz) of the power spectral density illustrated in FIG. 3C, and HF is the sum total of the power in the high frequency region (0.15 Hz to 0.4 Hz).

Herein, the HRV factors used for the evaluation include factors in the time domain and factors in the frequency domain. Known examples of the factors in the time domain include SDNN (standard deviation of normal—to normal intervals), RMSSD (root mean square of successive difference of RR intervals), and pNN50. Known examples of the factors in the frequency domain include total power serving as the sum total of the power in all the frequency domains, in addition to LF, HF, and LF/HF. In these factors, RMSSD, pNN50, and HF serve as indexes indicating parasympathetic nervous system activities. In addition, SDNN, the total power, and LF serve as indexes indicating the total activities of the sympathetic nervous system and the parasympathetic nervous system. LF/HF serves as an index indicating the activity of only the parasympathetic nervous system. As explained later, in the embodiment, the stress is detected by separately evaluating the sympathetic nervous system activity and the parasympathetic nervous system activity. Accordingly, the factor calculator 12 calculates, for example, LF, HF, and LF/HF as the HRV factors, outputs LF/HF to the first change detector 13 as the index indicating the sympathetic nervous system activity, and outputs HF to the second change detector 14 as the index indicating the parasympathetic nervous system activity.

Generally, LF includes information of the sympathetic nervous system activity and the parasympathetic nervous system activity. For this reason, it is widely executed to use LF/HF being a value acquired by dividing LF by HF indicating the power of the parasympathetic nervous system activity, as the index of the sympathetic nervous system activity. This example is also adopted herein to use LF/HF as the index of the sympathetic nervous system activity, but, observing LF enables easier intuitive understanding in the case of considering the power of the sympathetic nervous system activity. Although the numerical meaning thereof is little because LF is a value acquired by multiplying LF/HF by HF, using LF is also useful to intuitively observe sympathetic nervous system activities.

An example illustrated herein is an example in which the factor calculator 12 calculates LF, HF, and LF/HF. In the embodiment, it is possible to use other HRV factors enabling evaluation of activities of the sympathetic nervous system and the parasympathetic nervous system, as well as LF, HF, and LF/HF.

The first change detector 13 detects activity of the sympathetic nervous system of the subject from a chronological change of a first HRV factor indicating the sympathetic nervous system activity. For example, the first change detector 13 detects activity of the sympathetic nervous system of the subject by detecting that LF/HF of the time series has reached a peak value. The first change detector 13 can detect that LF/HF of the time series has reached a peak value, by detecting that, for example, LF/HF has increased over a rise threshold and thereafter has changed to decrease.

The second change detector 14 detects activity of the parasympathetic nervous system of the subject from a chronological change of a second HRV factor indicating the parasympathetic nervous system activity. For example, the second change detector 14 detects activity of the parasympathetic nervous system of the subject by detecting that HF of the time series has reached a peak value. The second change detector 14 can detect that HF of the time series has reached a peak value, by detecting that, for example, HF has increased over a rise threshold and thereafter has changed to decrease.

The stress detector 15 detects presence/absence of stress of the subject from a comparison result in the first change detector 13 and a comparison result in the second change detector 14.

The following is an explanation of a principle of stress detection by the stress detector 15. First, the following is an explanation of grasp of autonomous nervous system activities for grasp of stress. The inventors have researched emotions serving as human action/decision-making factors, and, in the process, have found similarity between emotional responses and physical responses in activity of the sympathetic nervous system.

Emotions themselves are self-preservation responses based on the instinct, and always exhibit negative responses. When responses of fright emotions of rats are mentioned as an example, the pupils of rats are dilated, the heart rate thereof increases, the blood pressure thereof increases, and they bristle, under the stimulus expected to cause fright. Bristling states are known as threat behaviors also in many mammals. On the other hand, human sympathetic nervous system responses also include increased heart rate, vasoconstriction, and contraction of arrector pili muscle. In other words, it can be said that these human sympathetic nervous system responses are similar to responses of fright emotions of rats. Autonomous nervous system act with no relation to human's intentions, and mainly act as vital homeostatic functions for visceral organs, such as the heart, the respiratory organs, the digestive organs, and the kidney. Autonomous nervous system is divided into sympathetic nervous system and parasympathetic nervous system. Sympathetic nervous system act for maintaining and improving motor functions required to avoid danger, called “fight or flight”. The purpose of responses of fright emotions of rats is also avoidance of danger. Based on this matter, the inventors reached the idea that fright emotional responses and the sympathetic nervous system responses might be exactly the same phenomena.

The sympathetic nervous system is activated by stimuli from the outside, and sympathetic nervous system activity is suppressed by activation of the parasympathetic nervous system. This is the essential part of autonomous nervous system activities. In addition, existence of living beings is maintained by keeping the balance (homeostasis) between the sympathetic nervous system and the parasympathetic nervous system.

Routinely, sympathetic nervous system is activated by stimuli constantly entering through the sensory organs from the environment, but the sympathetic nervous system activities are suppressed without any problem by activities of parasympathetic nervous system, and a state with little stress is maintained. However, the excited state of the sympathetic nervous system is maintained for stimuli deviating from the routine. The parasympathetic nervous system is not activated until the danger of the situation in which the human is placed is removed. This is a high stress state. Hereinafter, the explanation is continued with such similarity between emotional responses and physical responses in activity of the sympathetic nervous system used as an autonomous nervous system cause model of emotion.

In the Emotion Generation Model caused by SympAthetic nervous System Activation (:EGMsasa), all stimuli provided from the outside environment are supposed to be transmitted as emotion to the limbic system in the thalamus caused by the activation of the sympathetic nervous system. Regardless of consciousness and unconsciousness, stimuli from the outside environment should be constantly input, and the sympathetic nervous system must be constantly activated.

The stimulus that has reached the thalamus is simultaneously transmitted to the cortex path. In the cortex, the stimulus is analyzed. As a result of analysis, if the stimulus is determined as a routine stimulus that does not require particular attention, the parasympathetic nervous system is activated, and the sympathetic nervous system activities are suppressed. For example, stimuli such as environmental sound overflowing in daily life and environmental light including illumination are routine stimuli.

On the other hand, if the stimulus is an extraordinary stimulus, the parasympathetic nervous system is not activated. To continue to deal with the danger, the sympathetic nervous system is kept activation. The state of receiving such an extraordinary stimulus is a stress state. Mental health disorders, such as depression, are caused by long-term exposure to strong stress.

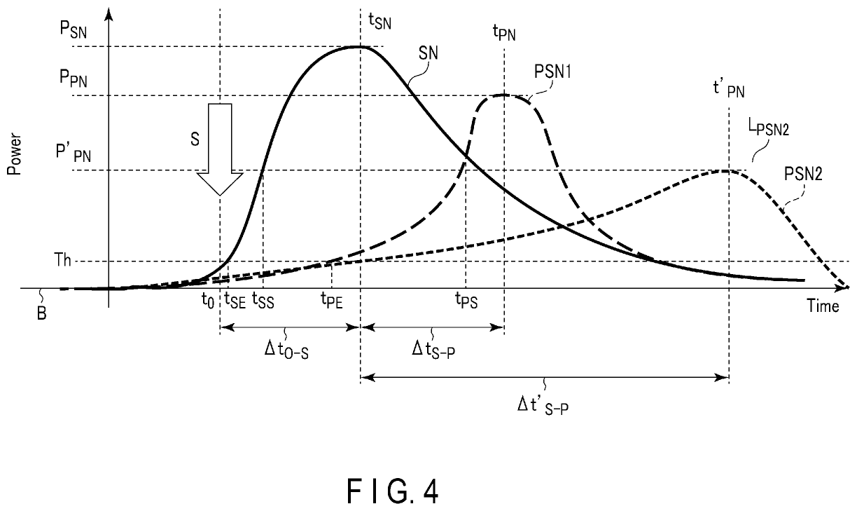

FIG. 4 is a diagram illustrating autonomic nervous system activities occurring by stimuli from outside, based on EGMsasa. The horizontal axis of FIG. 4 indicates the time, and the vertical axis indicates the power of the nervous system activity. The baseline B is, for example, a value of power of routine nervous system activity of a virtual subject. In addition, a curve SN in FIG. 4 illustrates chronological change of a value of power of the sympathetic nervous system activity. On the other hand, each of curves PSN1 and PSN2 in FIG. 4 indicates chronological change of a value of power of the parasympathetic nervous system activity. The curve PSN1 and the curve PSN2 indicate respective responses of the parasympathetic nervous system to different extraordinary stimuli.

According to EGMsasa, if a stimulus is provided from the outside, the sympathetic nervous system is constantly activated. The activated state of the sympathetic nervous system is suppressed by activity of the parasympathetic nervous system.

For example, if a stimulus S is provided at time to, the power SN of the sympathetic nervous system activity has a peak value P_{SN} at time t_{SN}. The peak value P_{SN} of the power SN of the sympathetic nervous system activity may change according to the intensity of the provided stimulus. The intensity of the stimulus S is proportional to the physical quantity thereof, if the stimulus S is a simple physical stimulus. In addition, the intensity of the stimulus S depends on the subject even if it is the same stimulus, and the intensity increases if it is a stimulus that the subject is weak at. If the intensity of the stimulus is high, and peak value P_{SN} increases. Accordingly, the workload of the sympathetic nervous system activity also increases, that is, the integrated value of the power SN of the sympathetic nervous system activity also increases. On the other hand, in the case of a sympathetic nervous system activity, peak time Δt_{0-S}=t_{SN}-t₀ until the power reaches the peak has a value conforming to unique response time (latent time) of the sensory organ regardless of the stimulus.

In contrast, because the parasympathetic nervous system is not activated until the danger of the situation in which the subject is placed is removed, the peak time until the power of the parasympathetic nervous system activity reaches the peak depends on not only the time at which the stimulus was provided, but also depends on the intensity of the stimulus. Specifically, if a weak stimulus is provided, the peak time Δt_{S-P}=t_{PN}-tₛₙ until the parasympathetic nervous system activity reaches the peak value P_{PN} is short as illustrated with the curve PSN1 in FIG. 4. If a strong stimulus is provided, the peak time Δt′_{S-P}=t′_{PN}-tₛₙ until the parasympathetic nervous system activity reaches the peak value P′_{PN} is long as illustrated with the curve PSN2 in FIG. 4. In addition, contrary to the sympathetic nervous system activity, the peak value of the power of the parasympathetic nervous system activity has a large value with a weak stimulus, and a small value with a strong stimulus. Specifically, with respect to a strong stimulus, the degree of activity thereof is small, or the parasympathetic nervous system is not activated, or hard to activate, until the danger is removed.

As described above, the stress state is a state in which the subject is under an extraordinary strong stimulus. In other words, the stress state is a state in which the time from detection of activity of the sympathetic nervous system to detection of activity of the parasympathetic nervous system is longer than the time from detection of activity of the sympathetic nervous to detection of activity of the parasympathetic nervous system in a case where a routine stimulus is provided. The time Δt_{S-P} from detection of activity of the sympathetic nervous system to detection of activity of the parasympathetic nervous system can be measured as a time difference between the time at which the power of the sympathetic nervous system activity reached the peak and the time at which the power of the parasympathetic nervous system activity reached the peak.

Based on the idea described above, the stress detector 15 detects a stress state by comparing the time Δt_{S-P} with a threshold. As another example, the stress detector 15 detects a stress state by detecting that the time elapsed from the time t_{SN} at which the power of the sympathetic nervous system activity reached the peak has exceeded the threshold. Herein, the threshold is time Δt_{S-P} in a case where a routine stimulus is provided, and set with time of about 200 ms to 300 ms as a standard. The threshold may be provided by measuring, for each subject, the time Δt_{S-P} in a case where a routine stimulus is provided.

Herein, the time used as an index for detection of the stress state is not limited to the time difference between the time at which the power of the sympathetic nervous system activity reached the peak and the time at which the power of the parasympathetic nervous system activity reached the peak. The time used as an index for detection of the stress state may be, for example, a time difference between the rise time of the power of the sympathetic nervous system activity and the rise time of the power of the parasympathetic nervous system activity. The rise times may be times t_{SE} and t_{PE} at which the powers of the respective nervous system activities reached a predetermined rise threshold Th, or may be times t_{SS} and t_{PS} of inflection points of the respective powers.

The outside stimulus acquisition unit 16 successively acquires pieces of information of outside stimuli to the subject. The stimuli are image stimuli and acoustic stimuli, etc., and are not particularly limited. In a case of acquiring image stimuli, the outside stimulus acquisition unit 16 includes a camera. Herein, the camera may be set to image the subject oneself. In addition, the camera may be mounted on, for example, the head of the subject to capture an image of a field of view of the subject. In addition, the camera may be an all-around camera capable of imaging not only the field of view of the subject but also the surroundings thereof. The camera may be a camera executing time-lapse imaging, not movie imaging. In addition, in a case of acquiring an acoustic stimulus, the outside stimulus acquisition unit 16 includes a microphone. When various events are classified into artificial and non-artificial ones, events serving as stressors are clearly artificial in many cases. In particular, it is considered that events serving as artificial stressors include many events relating to personal relations. Such events serving as artificial stressors can be determined by acquiring the sound at that time. The sound having a time stamp and positional information provides information as to when, where, and with whom the subject talked and what conversation was made. In addition, an accidental large event, such as a traffic accident, generates recordable large sound in itself. Such an event can also be determined by sound. In addition, picture display of an information device being viewed by the subject can be handled as an outside stimulus. In this case, the outside stimulus acquisition unit 16 acquires data of a screen being displayed at present from a display device displaying the screen. As another example, the outside stimulus acquisition unit 16 acquires data of the screen being displayed, by identifying the place that the user is observing by detection of line of sight using the camera. In addition, information dealt with as the outside stimulus also includes information relating to applications being used by the subject, such as information as to what operations have been made and what mails have been received. In this case, the outside stimulus acquisition unit 16 acquires necessary information from the relevant application. In addition, the outside stimulus acquisition unit 16 may be configured to acquire various situations in which the subject is placed and various events occurring around the subject.

The stimulus identification unit 17 identifies the stimulus supposed to provide stress to the subject at the time when the stress state is detected by the stress detector 15 as the stressor. The stimulus identification unit 17 also calculates the degree of influence of the stress provided to the subject by the stressor.

The stimulus supposed to provide stress to the subject is a stimulus acquired by the outside stimulus acquisition unit 16 just before detection of the stress state, that is, just before activity of the sympathetic nervous system at the time when the time Δt_{S-P} exceeded the threshold, that is, at the time to.

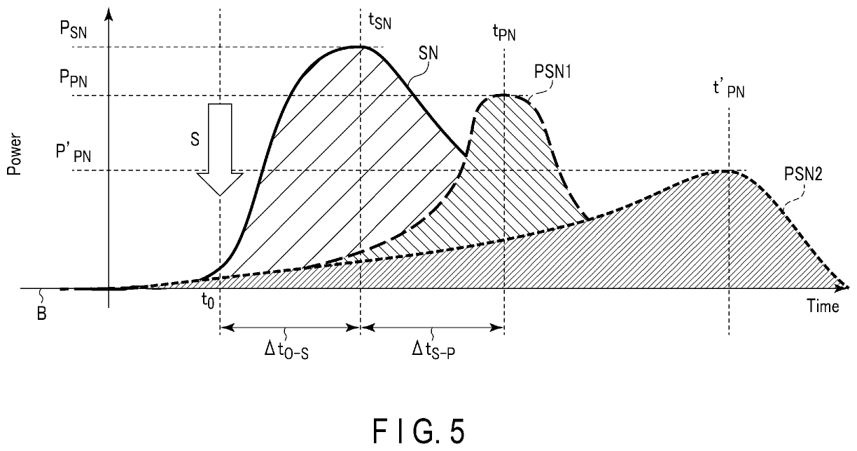

The degree of influence of the stimulus can be calculated from the workloads of the sympathetic nervous system activity and the parasympathetic nervous system activity, that is, the integrated value of the power SN of the sympathetic nervous system activity and the parasympathetic nervous system activity PSN. This is because the workload spent for dealing with the stimulus is proportional to the intensity of the stimulus serving as the stress. FIG. 5 is a diagram illustrating the sympathetic nervous system activity power SN, the parasympathetic nervous system activity power PSN1, and the parasympathetic nervous system activity power PSN2 painted in different hatching.

The controller 18 performs control to present information relating to the stress to the subject. For example, the controller 18 displays information, such as presence/absence of stress of the current subject, the name of the stressor, and the degree of influence provided by the stressor, on the display device. The information is not necessarily presented by display. The information may be presented by another method, such as output of sound. In addition, the controller 18 may perform control to store information for learning and the like.

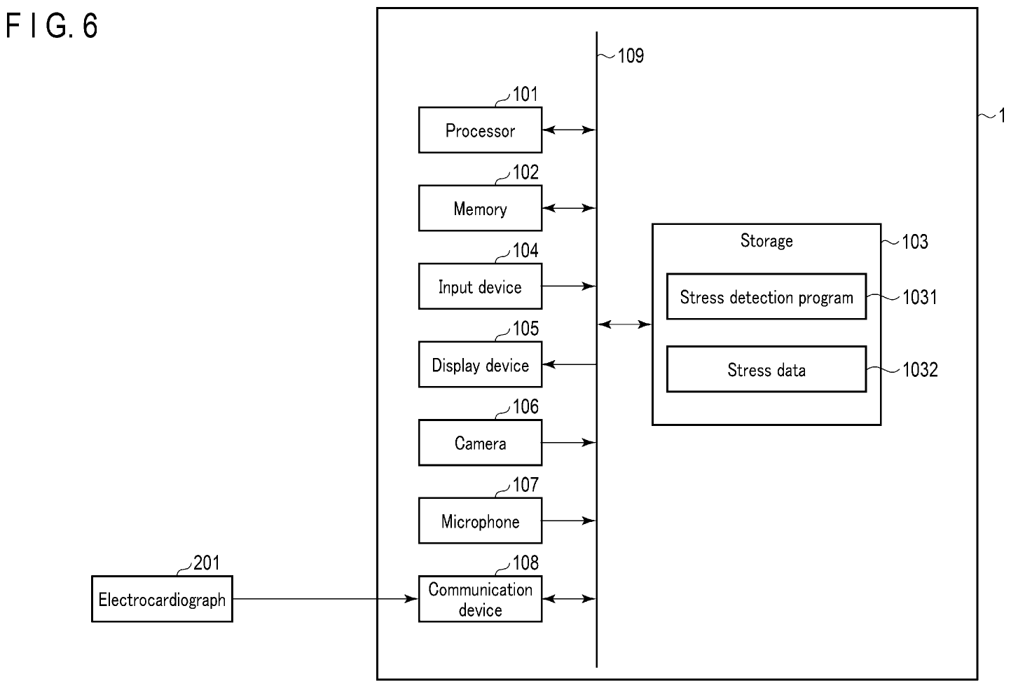

FIG. 6 is a diagram illustrating an example of a hardware configuration of the stress detection apparatus 1. The stress detection apparatus 1 may be a computer including, for example, a processor 101, a memory 102, a storage 103, an input device 104, a display device 105, a camera 106, a microphone 107, and a communication device 108, as hardware. The processor 101, the memory 102, the storage 103, the input device 104, the display device 105, the camera 106, the microphone 107, and the communication device 108 are connected to a bus 109. The stress detection apparatus 1 may be mounted on a terminal device, such as a personal computer (PC), a smartphone, and a tablet terminal.

The processor 101 is a processor controlling the entire operations of the stress detection apparatus 1. The processor 101 operates as the heartbeat acquisition unit 11, the factor calculator 12, the first change detector 13, the second change detector 14, the stress detector 15, the outside stimulus acquisition unit 16, the stimulus identification unit 17, and the controller 18, by executing a stress detection program stored in the storage 103, for example. The processor 101 is, for example, a CPU. The processor 101 may be an MPU, a GPU, an ASIC, or an FPGA or the like. The processor 101 may be a single CPU or the like, or a plurality of CPUs or the like.

The memory 102 includes a ROM and a RAM. The ROM is a non-volatile memory. The ROM stores a starting program or the like of the stress detection apparatus 1. The RAM is a volatile memory. The RAM is used as a working memory in processing in the processor 101, for example.

The storage 103 is a storage, such as a flash memory, a hard disk drive, and a solid-state drive. The storage 103 stores various programs executed by the processor 101, such as a stress detection program 1031. The storage 103 may operate as a storage unit and store stress data 1032. The stress data 1032 is data of the stress of each of subjects, and includes information, such as the date and time when the subject received the stress, the name of the stressor, and the degree of influence of the stressor.

The input device 104 is an input device, such as a touch panel, a keyboard, and a mouse. When the input device 104 is operated, a signal corresponding to the details of the operation is input to the processor 101 via the bus 109. The processor executes various types of processing in accordance with the signal.

The display device 105 is a display device, such as a liquid crystal display and an organic EL display. The display device 105 displays various images.

The camera 106 collects images as outside stimuli to the subject. The microphone 107 collects sound and environmental sound serving as the outside stimulus to the subject.

The communication device 108 is a communication device to communicate with external devices by the stress detection apparatus 1. The communication device 108 may include a communication device for wired communication or a communication device for wireless communication. The communication device 108 receives electrocardiographic data from the electrocardiograph 201 by, for example, communicating with the electrocardiograph 201. The electrocardiograph 201 is attached to the subject's wrist or chest or the like to collect chronological electrocardiographic data of the subject. The structure of the electrocardiograph 201 is not limited to any specific structure. For example, the electrocardiograph 201 may be mounted on a smartwatch.

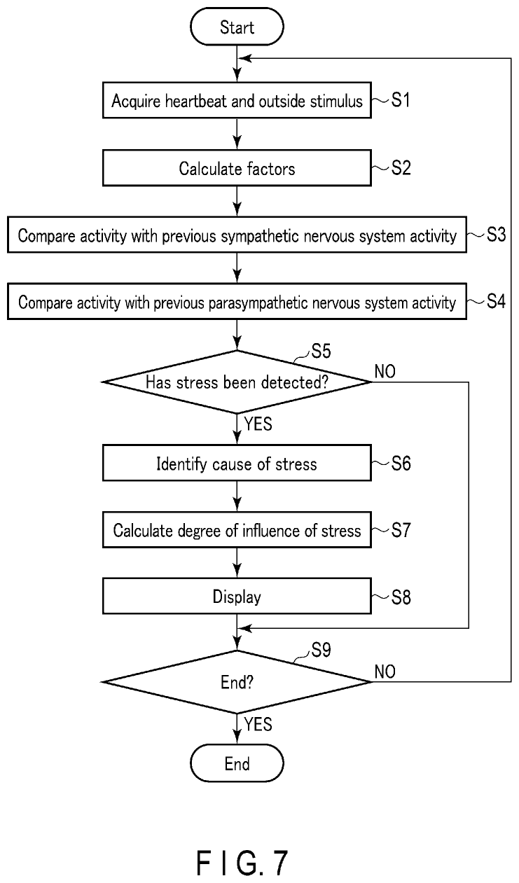

FIG. 7 is a flowchart illustrating operations of the stress detection apparatus 1. The process in FIG. 7 is executed by the processor 101.

At Step S1, the processor 101 acquires heartbeat data and information of the outside stimulus. With respect to the heartbeat data, the processor 101 converts the heartbeat data collected from the electrocardiograph 201 via the communication device 108 into RRI data as the heartbeat data, and stores the RRI data in the memory 102. With respect to the information of the outside stimulus, the processor 101 stores images collected with the camera 106 and sound collected with the microphone 107 in the memory 102.

At Step S2, the processor 101 calculates heart rate variability factor data from the heartbeat data. The processor 101 calculates, for example, HF serving as the second factor, and LF and LF/HF serving as the first factor from the RRI data.

At Step S3, the processor 101 detects the peak of the sympathetic nervous system activity by comparing the sympathetic nervous system activity serving as the first factor with the previous sympathetic nervous system activity. Specifically, the processor 101 compares the current LF/HF with the previous LF/HF.

At Step S4, the processor 101 detects the peak of the parasympathetic nervous system activity by comparing the parasympathetic nervous system activity serving as the second factor with the previous parasympathetic nervous system activity. Specifically, the processor 101 compares the current HF with the previous HF.

At Step S5, the processor 101 determines whether a stress state has been detected. As described above, the processor 101 detects a stress state by comparing the time Δt_{S-P} with the threshold. At Step S5, if a stress state is detected, that is, if it is determined that the time Δt_{S-P} is longer than the threshold, the process proceeds to Step S6. At Step S5, if no stress state is detected, that is, if it is determined that the time Δt_{S-P} is not longer than the threshold, the process proceeds to Step S9.

At Step S6, the processor 101 identifies the outside stimulus acquired at the time at which the stimulus was acquired previous to the time at which it was determined that the stress state was detected, that is, the outside stimulus acquired at the time earlier, by the peak time Δt_{0-S} of the sympathetic nervous system activity, than the peak time t_{SN} of the sympathetic nervous system activity at which it was determined that the stress state was detected, as the stimulus of the stressor. Unique sensory organ response time (latent time) may be used as the peak time Δt_{0-S} of the sympathetic nervous system activity.

Herein, the stressor different according to the details of the outside stimulus may be further identified. For example, if the identified stimulus is an image, the target that can be the stressor may be analyzed by identifying the object in the image by a method, such as object recognition in the image.

At Step S7, the processor 101 calculates the degree of influence provided by the stressor. As described above, the degree of influence provided by the stressor can be an integrated value of the powers of the sympathetic nervous system activity and the parasympathetic nervous system activity.

At Step S8, the processor 101 displays information relating to the stress on the display device 105. The information relating to the stress is information, such as presence/absence of stress of the user being the current subject, the name of the stressor, and the degree of influence provided by the stressor. At Step S8, the processor 101 may store information relating to the stress as the stress data 1032 in the storage 103.

At Step S9, the processor 101 determines whether to end the process. For example, it is determined to end the process if the power of the stress detection apparatus 1 is turned off, and/or if the user serving as the subject commands end of the process. At Step S9, if it is determined to end the process, the process of FIG. 7 is ended. At Step S9, if it is not determined to end the process, the process returns to Step S1. In this case, detection of the stress state is continued.

As described above, according to the present embodiment, by regarding the stress as an extraordinary stimulus on the basis of the EGMsasa, it is possible to detect presence/absence of the stress state from chronological changes of the sympathetic nervous system activity and the parasympathetic nervous system activity for the extraordinary stimulus. In addition, the embodiment enables identification of the stressor serving as a trigger of the stress state on the basis of the peak time of the sympathetic nervous system activity, by detecting presence/absence of the stress state based on the chronological changes of the sympathetic nervous system activity and the parasympathetic nervous system activity. In addition, the degree of influence provided by the stressor can also be calculated as workloads of the sympathetic nervous system activity and the parasympathetic nervous system activity. In this manner, the embodiment enables not only detection of the stress but also evaluation relating to protection of the mental health.

Herein, the explanation described above is based on the idea that stress can be regarded as an extraordinary stimulus based on EGMsasa. Experimental results acquired by the inventors are illustrated hereinafter.

In the experiment, the subject with an electrocardiograph attached to one's chest watched a display presenting a stimulus serving as a stressor. An image provided as the stimulus was an image in which an occult icon abruptly appeared in an everyday scene. The image was not seen by the subject before the experiment. In the experiment, another display was prepared, and the current time was displayed in an enlarged manner on the display. The image of the display was recorded by a movie camera to measure the presentation time of the stimulus.

In addition, in the experiment, electrocardiographic data collected by the electrocardiograph was transferred to a smartphone. After the experiment was finished, LF, HF, and LF/HF were calculated by FFT using the RRI value for 1 minute from the electrocardiographic data to determine the heart rate variability value during the experiment.

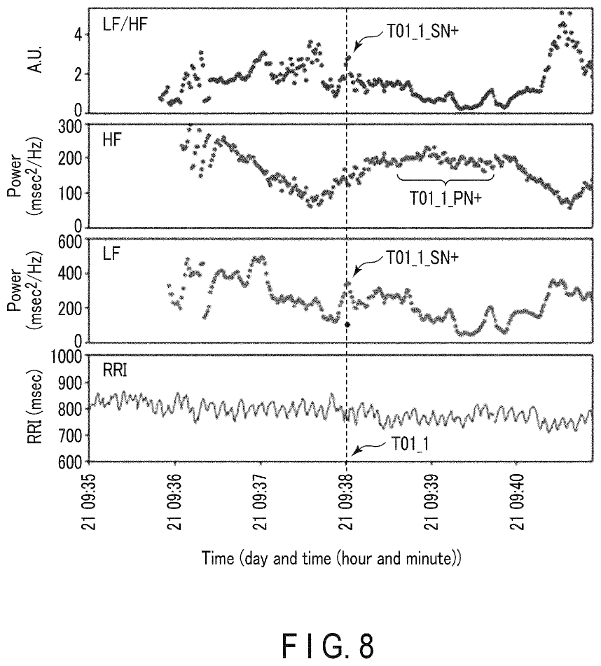

FIG. 8 is a diagram illustrating chronological changes of RRI, HF, LF, and LF/HF as experimental results. The horizontal axis of FIG. 8 indicates time. The time is indicated with the unit of date and time. In FIG. 8, a stimulus image was provided at the timing of 09:38 of the 21st day of the month. In addition, the graphs of FIG. 8 indicate RRI (milliseconds), the LF power (msec²/Hz), the HF power (msec²/Hz), and LF/HF, respectively, from the bottom.

As illustrated in FIG. 8, at the timing at which a stimulus image was provided, activity of the sympathetic nervous system was measured, that is, the peak of LF/HF was measured, as illustrated with T01_1_SN+. Although it was expected that sharp activation of the parasympathetic nervous system would occur after T01_1_SN+, gentle activation of the parasympathetic nervous system was observed, that is, the peak of HF was observed in the experiment, as illustrated with T01_1_PN+. This is considered to be the parasympathetic nervous system activity suppressing activity of the sympathetic nervous system due to the stimulus.

As described above, it was observed also in the experiment that the sympathetic nervous system was activated with a stimulus serving as a trigger and thereafter the parasympathetic nervous system was activated to suppress activity of the sympathetic nervous system. From this fact, it was verified that it was possible to detect a stress state on the basis of the EGMsasa.

### Modification

In the embodiment, the stress detection apparatus 1 detects presence/absence of a stress state in real time regardless of the degree of the outside stimulus. In contrast, the stress detection apparatus 1 may detect presence/absence of a stress state only in a case where an outside stimulus of a certain degree that can be a trigger of stress exists.

In addition, in the embodiment, the stress detection apparatus 1 detects presence/absence of a stress state, identifies the stressor, and calculates the degree of influence of the stressor on the basis of chronological changes of the sympathetic nervous system activity and the parasympathetic nervous system activity. In contrast, past detection results may be used for detection of a future stress state. For example, the stress detector 15 of the stress detection apparatus 1 may learn a relation between the stressor and the degree of influence, and determine that the subject is under the stress state, without determining presence/absence of a stress state, if the outside stimulus acquisition unit 16 acquires a stimulus similar to a past one. In addition, in this case, the stimulus identification unit 17 may determine that a stressor with the same degree of influence as that of the past one exists.

In addition, learning of the stressor and the degree of influence is not necessarily executed in the single stress detection apparatus 1, but may be executed in an upstream system of the stress detection apparatus 1. In this case, the upstream system can execute more statistical analysis based on pieces of information of the stressor and the degree of influence transferred from a number of stress detection apparatuses 1.

In addition, in the embodiment, the controller 18 is supposed to present information relating to the stress to the user serving as the subject. In contrast, the presentation destination of the information relating to the stress is not necessarily the subject oneself, but may be a person concerned with the subject. In this manner, the person concerned with the subject is enabled to take measures against the stress state of the subject early.

In addition, the instructions illustrated in the processing procedure illustrated in the embodiment described above may be executed on the basis of a program being software. A general-purpose computer system can obtain effects similar to effects of the stress detection apparatus described above by storing the program in advance and reading the program. The instructions described in the embodiment described above are recorded on a magnetic disk (such as a flexible disk and a hard disk), an optical disk (such as a CD-ROM, a CD-R, a CD-RW, a DVD-ROM, a DVD±R, a DVD±RW, and a Blu-ray (registered trademark) disc), a semiconductor memory, or a similar recording medium, as a program executable by a computer. The recording format thereof may be any format, as long as it is a recording medium readable by a computer or an integrated system. The computer is enabled to achieve operations similar to those of the stress detection apparatus according to the embodiment described above, by reading the program from the recording medium and executing the instructions described in the program with a CPU on the basis of the program. As a matter of course, the computer may acquire or read the program via a network in a case of acquiring or reading the program.

In addition, part of the processes to achieve the present embodiment may be executed by an OS (operating system) and/or database management software operating on the computer, or a middleware such as a network, on the basis of the instructions of the program installed in the computer or the integrated system from the recording medium.

In addition, the recording medium in the present embodiment is not limited to a medium independent of a computer or an integrated system, but also includes a recording medium storing or temporarily storing the program transmitted through a LAN and/or the Internet by download.

In addition, the number of the recording medium is not limited to one, but the recording medium according to the present embodiment also includes a case where the processes in the present embodiment is executed from a plurality of mediums, and the medium may have either structure.

The computer or the integrated system according to the present embodiment is one to execute each process in the present embodiment on the basis of the program stored in the recording medium, and may have either structure of a single apparatus, such as a personal computer and a microcomputer, and a system in which a plurality of apparatuses are connected via a network.

The computer in the present embodiment is not limited to a personal computer, but also includes an arithmetic processing unit and/or a microcomputer included in an information processing apparatus, and serves as a general term of apparatuses and/or devices capable of achieving the functions in the present embodiment with a program.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A stress detection apparatus comprising:
a processor including hardware configured to:
acquire heartbeat information of a subject;
calculate a heart rate variability factor from the heartbeat information;
detect change of an activity of sympathetic nervous system of the subject, based on the heart rate variability factor;
detect change of an activity of parasympathetic nervous system of the subject, based on the heart rate variability factor;
detect presence/absence of a stress state of the subject, based on time from detection of the change of the sympathetic nervous system to detection of the change of the activity of the parasympathetic nervous system;
acquire outside stimuli to the subject; and
identify a stressor that has provided the stress state to the subject from the acquired outside stimuli, based on timing at which the change of the sympathetic nervous system has been detected if the stress state is detected.

2. The stress detection apparatus according to claim 1, wherein the processor detects that the subject is in the stress state if the time from detection of the change of the sympathetic nervous system to detection of the change of the activity of the parasympathetic nervous system is longer than a threshold.

3. The stress detection apparatus according to claim 1, wherein the processor further calculates a degree of influence of the stressor on the subject, based on workloads of the sympathetic nervous system and the parasympathetic nervous system.

4. The stress detection apparatus according to claim 1, further comprising:
a storage storing information relating to stress provided to the subject.

5. The stress detection apparatus according to claim 4, wherein
the information relating to stress includes information of the stressor provided to the subject, and
the processor detects presence/absence of the stress state, based on the acquired outside stimuli and the information of the stressor.

6. The stress detection apparatus according to claim 1, wherein the processor further presents information relating to stress provided to the subject.

7. The stress detection apparatus according to claim 1, wherein
the processor detects the change of the sympathetic nervous system of the subject, based on chronological change of a ratio of a low frequency domain component of a frequency domain to a high frequency domain component of the frequency domain of heartbeat intervals calculated from the heartbeat information, and detects the change of the parasympathetic nervous system of the subject, based on chronological change of the high frequency domain component of the frequency domain of the heart rate variability factors calculated from the heartbeat information.

8. A stress detection method comprising:
acquiring heartbeat information of a subject;
calculating a heart rate variability factor from the heartbeat information;
detecting change of sympathetic nervous system of the subject, based on the heart rate variability factor;
detecting change of parasympathetic nervous system system of the subject, based on the heart rate variability factor;
detecting presence/absence of a stress state of the subject, based on time from detection of the change of the sympathetic nervous system to detection of the change of the parasympathetic nervous system;
acquiring outside stimuli to the subject; and
identifying a stressor that has provided the stress state to the subject from the acquired outside stimuli, based on timing at which the change of the sympathetic nervous system has been detected if the stress state is detected.

9. A computer-readable non-transitory storage medium storing a stress detection program to cause a computer to execute:
acquiring heartbeat information of a subject;
calculating a heart rate variability factor from the heartbeat information;
detecting change of sympathetic nervous system of the subject, based on the heart rate variability factor;
detecting change of parasympathetic nervous system of the subject, based on the heart rate variability factor;
detecting presence/absence of a stress state of the subject, based on time from detection of the change of the sympathetic nervous system to detection of the change of the parasympathetic nervous system;
acquiring outside stimuli to the subject; and
identifying a stressor that has provided the stress state to the subject from the acquired outside stimuli, based on timing at which the change of the sympathetic nervous system has been detected if the stress state is detected.
